(19) United States
(12) Patent Application Publication
(10) Pub. No.: US 2013/0281540 A1
(43) Pub. Date: Oct. 24, 2013

(54) Method for the Subcutaneous Administration of Parenteral Medications

(71) Applicant: Borislav M. MRAVKOV, Wilmington, NC (US)

(72) Inventor: Borislav M. MRAVKOV, Wilmington, NC (US)

(21) Appl. No.: 13/851,950

(22) Filed: Mar. 27, 2013

Related U.S. Application Data
(60) Provisional application No. 61/615,912, filed on Mar. 27, 2012.

(51) Int. Cl. A61M 5/00

(52) U.S. Cl. CPC A61M 5/00
USPC 514/653

(57) ABSTRACT

The invention presently disclosed is a method for the administration of parenteral medication to a person via subcutaneous delivery designed and intended to facilitate the quick, safe and effective administration of such medication, the method having implications in the treatment of a host of conditions and ailments. As conceived, the inventor contemplates the method having particularized utility in the administration of epinephrine to a person suffering from the life-threatening condition of anaphylaxis.

### CROSS-REFERENCE TO RELATED APPLICATIONS

This application claims the benefit of priority to U.S. Provisional Application No. 61/615,912 filed Mar. 27, 2012. The content of U.S. Provisional Application No. 61/615,912 is incorporated by reference herein in its entirety.

### STATEMENT REGARDING FEDERALLY SPONSORED RESEARCH OR DEVELOPMENT

Not Applicable.

### THE NAMES OF THE PARTIES TO A JOINT RESEARCH AGREEMENT

Not Applicable.

### INCORPORATION-BY-REFERENCE OF MATERIAL SUBMITTED ON A COMPACT DISC

Not Applicable.

### BACKGROUND OF THE INVENTION

(a) Field of the Invention

The present invention is in the technical field of methods of administration of medication to a subject. More particularly, the present invention is in the technical field of methods of administration of parenteral medication to a subject. Still more particularly, the present invention is in the technical field of methods of subcutaneous administration of parenteral medication to a subject.

(b) Background Art

Anaphylaxis is a life threatening condition that affects people having severe allergies to a specific chemical or compound, the specific triggering chemical or compound often being encountered by a person in an often otherwise innocuous setting or context such as ingesting of foods or medications. Anaphylaxis is further defined as a serious allergic reaction that is rapid in onset and capable of causing death. It typically presents in a number of symptoms including an itchy rash, throat swelling, and low blood pressure and the condition may be caused by a wide variety of allergens including, but not limited to insect bites, foods, and medications—the condition is typically diagnosed based on the presenting symptoms and signs. On a pathophysiologic level, anaphylaxis results from the excessive release of mediators from certain types of white blood cells triggered either by immunologic or non-immunologic mechanisms. In the present state of the art, the primary treatment of anaphylaxis is injection of epinephrine (adrenaline), with other measures being complementary. Worldwide 0.05-2% of people are estimated to have anaphylaxis at some point in their life and rates appear to be increasing.

As stated above, injection of epinephrine is the primary treatment for anaphylaxis with no known contraindication to its use. In the present state of the art, known methods of treatment include the delivery of epinephrine via intravenous, intramuscular or subcutaneous injections. Far and away, the prevailing method of administering epinephrine is via intramuscular injection to the mid-anterolaterial thigh as soon as possible upon presentment of anaphylactic symptoms. The injection may be repeated every 5 to 15 minutes if there is insufficient patient response—a second dose is often required in many episodes of anaphylaxis with more than two doses rarely required. In the context of the prevailing treatment at the mid-anterolaterial thigh situs, the intramuscular route is preferred over subcutaneous administration because the latter is suspected to have delayed absorption at this situs, thus compromising speed, efficacy, and reliability. The most commonplace self-treatment option available to persons subject to anaphylaxis is the use of intramuscular auto-injection devices such as EpiPen®, the current industry standard in this respect.

Treatment of anaphylaxis via the industry-standard intramuscular injection to the mid-anterolateral thigh requires preparation of a hypodermic needle in advance of administration of the epinephrine dosage and, as such, is a process requiring 10-15 seconds to complete. Dependent upon the severity of the anaphylaxis and/or anaphylactic shock experienced by a person, the 10-15 seconds necessary in preparation and administration of a remedy are precious seconds lost and, in extreme cases, may be the difference between the life and death of the person. Beyond this procedural delay in the treatment of anaphylaxis via the industry-standard intramuscular injection to the mid-anterolateral thigh is a secondary physiological delay. Upon intramuscular injection to the mid-anterolateral thigh, the epinephrine dosage must travel approximately one (1) meter within the person's body before reaching the systemic circulation, a physiological journey that further delays the therapeutic effects of the dosage and thereby further endangers the life of the anaphylactic person.

The inventor attributes the industry affinity for treatment of anaphylaxis via the intramuscular injection to an industry perception that intramuscular administration, as compared to subcutaneous administration, is critical for the quick absorption of epinephrine which is, therefore, critical in achieving quick therapeutic effects from a dosage injected. There is further industry perception that subcutaneous delivery of epinephrine results in absorption that is too slow to be useful in the treatment of a medical emergency such as anaphylaxis—this latter perception is predicated upon the fact that that epinephrine is a local vasoconstrictor and, therefore, can slow its own absorption. Despite the above stated perceptions to the contrary, the reality of the art is that many attempts at intramuscular injection of epinephrine to the mid-anterolateral thigh are actually subcutaneous injects occasioned by the a body fat content of the subject person that is too great to allow the hypodermic needle to transverse the subcutaneous tissues during administration. Further, it is know in the art that the intentional practice in many emergency rooms is to administer epinephrine via subcutaneous injection at the mid-anterolateral thigh of an anaphylactic subject.

The state of the art is replete with devices and methods for the treatment of anaphylaxis through the intramuscular injection of epinephrine in a subject person. That said, the art would be greatly improved by the introduction of a method that improves upon the speed and safety in administration of epinephrine to a subject while maintaining or improving a minimum standard of effectiveness and reliability in implementation and ultimate delivery of the epinephrine dosage to the systemic circulation. Namely, the inventor believes the present state of the art suffers from misperceptions regarding the preferred situs of injection being in the limbs of a subject person and further suffers from misperceptions as to the supreme efficiency and advantage of intramuscular injection of epinephrine, a process rendering the use of a hypodermic needle a necessity.

### BRIEF SUMMARY OF THE INVENTION

The invention presently disclosed is a method for the administration of parenteral medication to a person via subcutaneous delivery designed and intended to facilitate the quick, safe and effective administration of such medication, the method having implications in the treatment of a host of conditions and ailments. As conceived, the inventor contemplates the method having particularized utility in the administration of epinephrine to a person suffering from the life-threatening condition of anaphylaxis.

In its broadest embodiment, the now disclosed inventive method is a method of administration of a parenteral medication to a person wherein the parenteral medication is administered via means of subcutaneous delivery at a situs in the upper torso area of the person. In more specific context, the inventor contemplates the administration of a parenteral medication to a person wherein the parenteral medication is administered via means of subcutaneous delivery at a situs proximate to and beneath the clavicle or proximate to the sternum. Still more specifically, the inventor contemplates the administration of a parenteral medication to a person wherein the parenteral medication is administered without needle and under force of compressed gas causing a concentration of medication the penetrate the person's skin and selectively deposit in the subcutaneous tissue at a situs proximate to and beneath the clavicle or proximate to the sternum.

It is an objective of the now disclosed inventive method to provide for the quicker delivery of epinephrine to the systemic circulation. The presently favored method of treatment of anaphylaxis is via intramuscular injection to the mid-anterolaterial thigh, a method that due to preparation of a hypodermic needle required for intramuscular injection and use of the same takes 10-15 seconds to complete. The presently disclosed inventive method enables subcutaneous delivery of epinephrine instantaneously in its preferred embodiment. Further, upon administration, an intramuscular injection to the mid-anterolaterial thigh must travel approximately one (1) meter within the body to reach the systemic circulation, whereas the now disclosed inventive method provides for subcutaneous administration at a site in the upper torso, including, but not limited to, a situs proximate to and beneath the clavicle or proximate to the sternum, meaning that the now disclosed administered medication must travel only 5-10 centimeters to the systemic circulation. Also hastening introduction of the epinephrine dosage to the systemic circulation during utilization of the now disclosed inventive method is the fact that subcutaneous administration at a site proximate to and beneath the clavicle or proximate to the sternum instantaneously introduces the dosage to the lower neck and/or upper thoracic venous and lymphatic vessels that drain faster than vessels in the limbs due to negative pressures in the chest and upper torso during inspiration.

It is a further objective of the now disclosed inventive method to provide a targeted and direct application of an epinephrine dosage to a person experiencing anaphylactic shock in such way that therapeutic effects are immediately realized and, in the alternative and due to the possibility of delayed absorption, a time-release effect of a residual dosage is realized, thus providing continued protection against the late effects of an episode of anaphylactic shock.

It is a still further objective of the now disclosed inventive method that epinephrine and other comparable parenteral medications be quickly administered and delivered to the systemic circulation without compromising safety of administration in the process. Specifically, subcutaneous administration of a parenteral medication at a site in the upper torso, including, but not limited to, a situs proximate to and beneath the clavicle or proximate to the sternum, without use of a needle is desirable due to the fact that no major subcutaneous organs exist in the upper torso/chest area and, therefore, subcutaneous methods of delivery are safely used whereas utilization of a hypodermic needle endangers a variety of internal organs including, but not limited to the lungs, heart, vena cava, aorta, thyroid, etc. For this reason, needle-less subcutaneous administration is a highly favored method of administration in this region of the human anatomy.

It is a further objective of the now disclosed inventive method to generally introduce the concept of administration of an epinephrine dosage by needle-less means and, specifically, by means under pressure of compressed gas forcing a dosage of medication directly through the skin surface to selectively deposit in the subcutaneous tissues. While the inventor admits of devices that exist that accomplish this feat, the inventor is unaware in the prior art of any devices specifically designed to accomplish this feat particular to an epinephrine dosage in the treatment of anaphylaxis and, further, is unaware in the prior art of any devices or methods of using the same that target administration upon the upper torso to leverage the advantages of subcutaneous injection in that region and the concomitant benefits of expedited introduction of the medication to the systemic circulation.

Specifically, the inventor believes that the now disclosed inventive method marks an improvement upon the known methods of treatment of anaphylaxis, including but not limited to self-treatment via the use of intramuscular auto-injection devices such as the industry-standard EpiPen®, by introducing marked improvements in (i) the speed of introduction of epinephrine to the systemic circulation, (ii) the safety in administration of epinephrine, (iii) the effectiveness of administration of epinephrine and (iv) the reliability with which epinephrine is administered and delivered to the systemic circulation. It is the further belief of the inventor that the now disclosed inventive method has application beyond the treatment of anaphylaxis and the subcutaneous delivery of epinephrine, specifically in that the inventive method has like applicability in the effective and equivalent treatment of any condition requiring the delivery of a medication to the systemic circulation.

For purposes of the now disclosed inventive method, the inventor defines the term “proximate” when utilized with regard to the clavicle to mean any situs beneath the right or left clavicle and located within five (5) inches, and preferably within three (3) inches, of the right or left clavicle, as the case may be. For purposes of the now disclosed inventive method, the inventor defines the term “proximate” when utilized with regard to the sternum to mean any situs to the right or left of the sternum wherein the situs is located within three (3) inches of the sternum and preferable within three (3) inches of the upper third (⅓) of the sternum.

For purposes of the now disclosed inventive method, the inventor defines the term “needle-less” to mean any device apparatus, device, means, or method in which a needle is neither a component nor a contemplated whereby a medication may be administered to an individual. While the term “needle-less” as utilized by the inventor merely contemplates the absolute absence of a needle, the term is expressly contemplated to include, but not be limited to, the following modes of administration of a medication: non-invasive transdermal administration, transdermal diffusion, invasive transdermal administration under force of a propellent such as a pressurized gas, iontophoretic administration, fluid jet delivery, or other method of administration not utilizing a needle.

For purposes of the now disclosed inventive method, the inventor expressly defines the terms “administer”, “administers”, and “administration” to include both introduction of the parenteral medication to the systemic circulation per the now disclosed method (i) by a third-party medical provider to an afflicted person, or otherwise, (ii) self-administration by the afflicted person.

### BRIEF DESCRIPTION OF THE SEVERAL VIEWS OF THE DRAWING(S)

FIG. 1 is a flowchart depicting a preferred embodiment of the now disclosed inventive method.

FIG. 2 is a perspective view of a preferred embodiment of the now disclosed inventive method in action.

### DETAILED DESCRIPTION OF THE INVENTION

In the preferred embodiment and best mode, the now disclosed inventive method comprises administration of epinephrine or other parenteral medication to a person needing treatment for anaphylaxis wherein the epinephrine or other parenteral medication is administered via means of subcutaneous delivery at a situs proximate to and beneath the clavicle or proximate to the sternum. More specifically, the inventor contemplates the means of subcutaneous delivery in the preferred embodiment to be without needle and under force of compressed gas causing a concentration of medication the penetrate the person's skin and selectively deposit in the subcutaneous tissue at a situs proximate to and beneath the clavicle or proximate to the sternum.

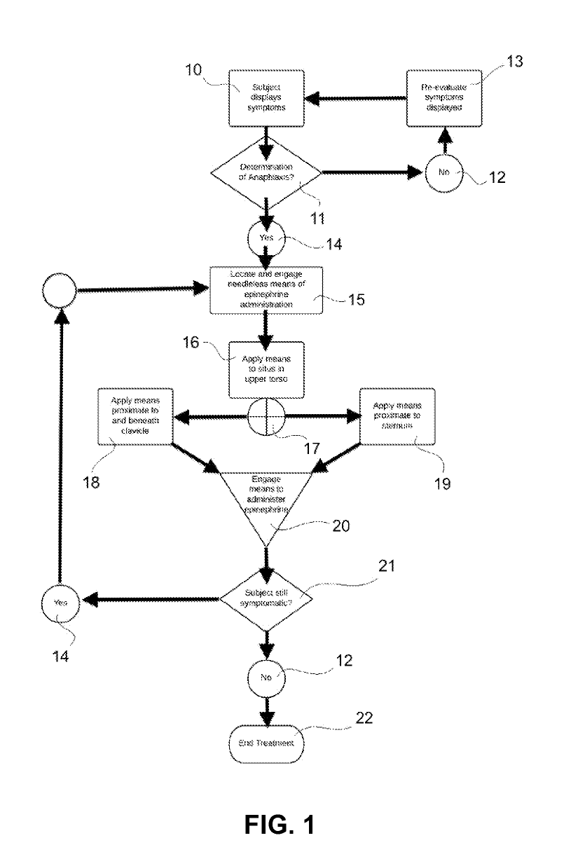

Referring now to the invention in more detail, in FIG. 1 there is shown a preferred embodiment of the now disclosed inventive method, the embodiment comprising the onset of symptoms 10 in a person and an initial determination of anaphlyaxis 11 resulting in either an affirmative 14 or negative 12 diagnosis. There is further shown a first alternative action 13 in the event of a negative 12 diagnosis and a second alternative action 15 in the event of an affirmative 14 diagnosis. Upon a positive 14 diagnosis, there is shown the now disclosed method of treatment, comprising the step of locating and engaging needle-less means of epinephrine administration 15, followed by application of the means at a situs in the upper torso of the individual 16. There is further depicted a first option 18 in which the means is applied to a situs proximate to and beneath the clavicle and a second option 19 wherein the means is applied proximate to the sternum. Upon user selection of either the first option 18 or the second option 19, there is depicted the step wherein the means is engaged and utilized to actually administer the epinephrine 20. Still further, there is depicted a secondary determination 21 to be assessed and answered in either the affirmative 14 or the negative 12. In the event of a negative 12 response to the secondary determination 21, the subject has been apply treated and the method is at a terminus 22. However, in the event of an affirmative 14 response to the secondary determination 21, the subject requires further treatment and the method begins anew with the step of locating and engaging needle-less means of epinephrine administration 15.

With regard to the preferred embodiment depicted in FIG. 1, the step of locating and engaging needle-less means of epinephrine administration 15 expressly contemplates that the needle-less means located and engaged will be a device that contains at least a single dose of epinephrine and that is particularly designed to deliver the dose contained via invasive transdermal administration under force of a propellent such as a pressurized gas or fluid jet delivery. Accordingly upon the step wherein the means is engaged and utilized to actually administer the epinephrine 20, the dose of epinephrine is forcibly passed through the skin at high pressure and without the use of a needle, thereby selectively depositing in the subcutaneous tissues.

It is contemplated that the manner and process of using the disclosed inventive method would entail administration of the parenteral medication per the now disclosed method by a third-party medical provider to an afflicted person, or otherwise, self-administration by the afflicted person. It is further contemplated that the employed means of subcutaneous delivery may be hypodermic needle or such otherwise needle-less means as may be known in the art, including, but not limited to, under force of compressed gas causing a concentration of medication the penetrate the person's skin and selectively deposit in the subcutaneous tissue at the situs of administration. It is further contemplated that the manner and process of use for the now disclosed inventive method would likely entail the treatment of anaphylaxis in a person by means of the administration of epinephrine by means of the now disclosed method and, in fact, this was the initial application the inventor had in mind when conceiving the inventive method.

Again while the preferred embodiment of the now disclosed invention as depicted in FIG. 1 clearly contemplates the use of epinephrine in the treatment of anaphylaxis, the contemplation is that the method itself may be applicable to any of a host of parenteral medications in treatment of any of a host of conditions.

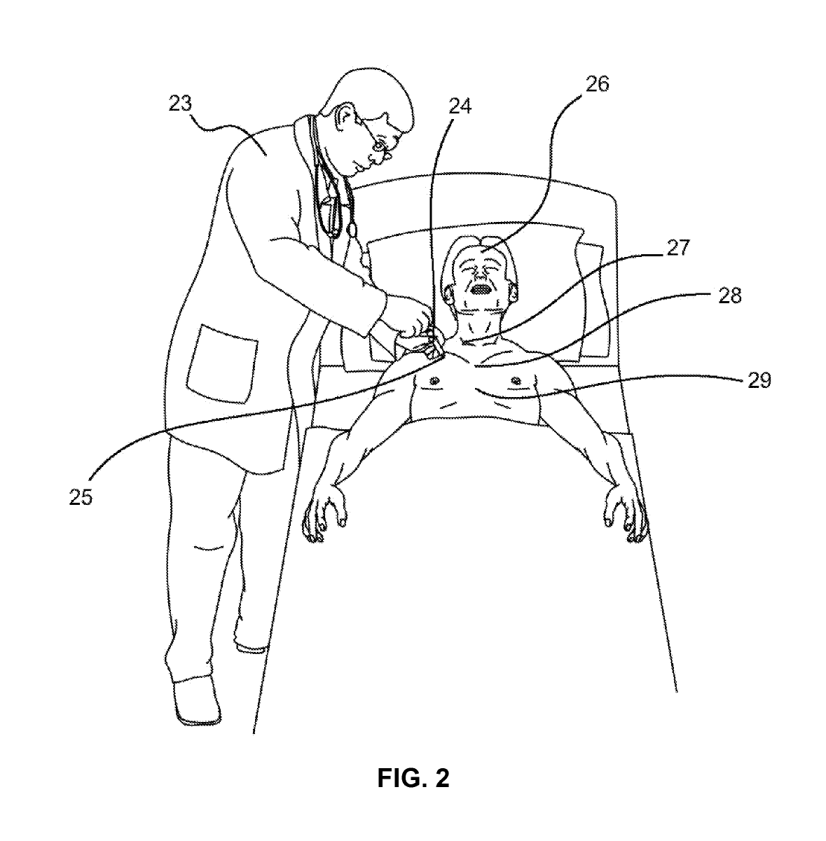

Referring now to the inventive method in more detail, in FIG. 2 there is shown a perspective view of the inventive method in action, the view depicting a doctor 23 administering a medicine via the inventive method to a subject patient 26. In still more detail, FIG. 2 depicts the preferred embodiment and best mode of the now disclosed inventive method in action, comprising the administration of epinephrine or other parenteral medication via means 24 of subcutaneous delivery at a situs 25 in the upper torso 29 of a patient 26, the situs 25 being proximate to and beneath the clavicle 27 of the patient 26. As before stated with regard to the preferred embodiment and best mode, the situs 25 of administration in the upper torso 29 of the patient 26 could alternatively have been located proximate to the sternum 28 of the patient 26, despite that this alternative location of administration is not presently illustrated in FIG. 2. Further, one of ordinary skill in the industry can appreciate that the depicted means 24 of subcutaneous delivery at the situs 25 may be hypodermic needle or such otherwise needle-less means as may be known in the art, including, but not limited to, under force of compressed gas causing a concentration of medication the penetrate the person's skin and selectively deposit in the subcutaneous tissue at the situs of administration.

Reference throughout the specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout the specification may, but do not necessarily, refer to the same embodiment.

Furthermore, the described features, advantages, and characteristics of the invention may be combined in any suitable manner in one or more embodiments. One skilled in the relevant art will recognize that the invention can be practiced without one or more of the specific features or advantages of a particular embodiment. In other instances, additional features and advantages may be recognized in certain embodiments that may not be present in all embodiments of the invention.

It is understood that the above described embodiments are only illustrative of the application of the principles of the present invention. The present invention may be embodied in other specific forms without departing from its spirit or essential characteristics. The described embodiment, including the best mode, is to be considered in all respects only as illustrative and not restrictive. The scope of the invention is, therefore, indicated by the appended claims, if any, in conjunction with the foregoing description.

While the foregoing written description of the invention enables one of ordinary skill to make and use what is considered presently to be the best mode thereof, those of ordinary skill will understand and appreciate the existence of variations, combinations, and equivalents of the specific embodiment, method, and examples herein. The invention should therefore not be limited by the above described embodiment, method, and examples, but by all embodiments and methods within the scope and spirit of the invention.

I claim:

1. A method of administration of a parenteral medication to a person, the method comprising subcutaneous delivery of the parenteral medication to a selective situs in the upper torso of the person.

2. The method of claim 1 wherein the selective situs in the upper torso of the person is proximate to and beneath the clavicle.

3. The method of claim 2 wherein the subcutaneous delivery of the parenteral medication is accomplished via a needle-less drug delivery means.

4. The method of claim 3 wherein the needle-less drug delivery means subcutaneously injects a dosage of parenteral medication under force of compressed gas causing the dosage to penetrate the skin and selectively deposit in the subcutaneous tissue.

5. The method of claim 4 wherein the parenteral medication is a dosage of epinephrine.

6. The method of claim 2 wherein the parenteral medication is a dosage of epinephrine.

7. The method of claim 1 wherein the selective situs in the upper torso of the person is proximate to the sternum.

8. The method of claim 7 wherein the subcutaneous delivery of the parenteral medication is accomplished via a needle-less drug delivery means.

9. The method of claim 8 wherein the needle-less drug delivery means subcutaneously injects a dosage of parenteral medication under force of compressed gas causing the dosage to penetrate the skin and selectively deposit in the subcutaneous tissue.

10. The method of claim 9 wherein the parenteral medication is a dosage of epinephrine.

11. The method of claim 7 wherein the parenteral medication is a dosage of epinephrine.

12. The method of claim 1 wherein the parenteral medication is a dosage of epinephrine.

13. The method of claim 12 wherein the subcutaneous delivery of the parenteral medication is accomplished via a needle-less drug delivery means.

14. The method of claim 13 wherein the needle-less drug delivery means subcutaneously injects a dosage of parenteral medication under force of compressed gas causing the dosage to penetrate the skin and selectively deposit in the subcutaneous tissue.

15. The method of claim 1 wherein the subcutaneous delivery of the parenteral medication is accomplished via a needle-less drug delivery means.

16. The method of claim 15 wherein the needle-less drug delivery means subcutaneously injects a dosage of parenteral medication under force of compressed gas causing the dosage to penetrate the skin and selectively deposit in the subcutaneous tissue.

17. A method of administration of parenteral medication to a person, the method comprising—
selection of a parenteral medication;
coupling of the selected parenteral medication with means of subcutaneous delivery;
application of the means of subcutaneous delivery to a selective situs in the upper torso of the person; and
activation of the means of subcutaneous delivery to cause the subcutaneous delivery of the parenteral medication to the selective situs in the upper torso of the person.

18. The method of claim 17 wherein the parenteral medication is a dosage of epinephrine.

19. The method of claim 17 wherein the selective situs in the upper torso of the person is proximate to and beneath the clavicle or is proximate to the sternum.

20. The method of claim 19 wherein the parenteral medication is a dosage of epinephrine.

21. The method of claim 17 wherein the means of subcutaneous delivery is a needle-less drug delivery means.

22. The method of claim 21 wherein the needle-less drug delivery means subcutaneously injects a dosage of parenteral medication under force of compressed gas causing the dosage to penetrate the skin and selectively deposit in the subcutaneous tissue.

23. The method of claim 22 wherein the parenteral medication is a dosage of epinephrine.

24. The method of claim 21 wherein the parenteral medication is a dosage of epinephrine.

25. A method of administration of epinephrine via subcutaneous injection to a person experiencing anaphylaxis, the method comprising—
storage of a dosage of epinephrine in a needle-less drug delivery apparatus;
application of the needle-less drug delivery apparatus to a situs proximate to the clavicle or sternum in the upper torso of a person; and
subcutaneous injection of the dosage of epinephrine under force of compressed gas causing the dosage to penetrate the skin and selectively deposit in the subcutaneous tissue.
